(19) United States
(12) Patent Application Publication
(10) Pub. No.: US 2008/0183153 A1
(43) Pub. Date: Jul. 31, 2008

(54) Enteral Feeding Tube Connector

(75) Inventor: Thomas Frederick ENNS, Mississauga (CA)

Correspondence Address: WILLIAM J. SAPONE;COLEMAN SUDOL SAPONE P.C., 714 COLORADO AVENUE, BRIDGE PORT, CT, 06605, US

(73) Assignee: BENLAN, INC., Oakville, ON (CA)

(21) Appl. No.: 11/669,502

(22) Filed: Jan. 31, 2007

(51) Int. Cl. A61M 39/10; A61M 25/00

(52) U.S. Cl. 604/533; 604/523

(57) ABSTRACT

An enteral connector includes a body defining a passageway therethrough. The body has an inlet segment defining an inlet opening of the passageway, suitable for connection of an enteral feeder such as an enteral feeding extension set or an enteral-only syringe. The body also has an outlet segment including a stem projecting from the inlet segment and a frustoconical tip defining an outlet opening of the passageway, for connecting to an enteral feeding tube or a tube of an enteral feeding extension set. The enteral connector also includes a cap having an external cover for covering the outside of the inlet segment and a plug integral with and extending from the external cover for inserting into the inlet opening. The cap is connected to and integral with the body by an intermediate flexible neck.

### FIELD OF THE INVENTION

The present invention relates to connection of enteral feeding tubes and feeding sets containing food or medication.

### BACKGROUND OF THE INVENTION

Enteral tube feeding is known for feeding patients such as infants or children who are unable to ingest foods orally. The enteral tube is inserted either orally or nasally, through the oesophagus and into the stomach and is used to deliver nutritive liquid feed to the stomach of the infant or child for digestion. One end of the tube remains external to the patient and is used for connecting an enteral feeding extension set or syringe for delivery of the liquid feed. The opposing end of the tube is located in the stomach of the patient to which the liquid feed is delivered.

A variety of enteral feeding tubes including polyurethane and silicone tubes are used for more than a single delivery of liquid feed to the patient to avoid problems associated with frequent insertion and extraction of feeding tubes. For example, polyurethane feeding tubes are generally used for up to about 30 days while silicone feeding tubes can be used even longer.

A connector at the end of the feeding tube that is external to the patient is used for connecting the enteral feeding extension set or syringe to the feeding tube. Similarly, a connector at the end of the enteral feeding extension set is used for connecting the enteral feeding tube to enteral feeding equipment or a syringe. Improvements in enteral connectors are driven by the desire for increased safety, usability and improved performance. It is therefore desirable to provide an improved enteral connector.

### SUMMARY OF THE INVENTION

According to one aspect, there is provided an enteral connector that includes a body defining a passageway therethrough. The body has an inlet segment defining an inlet opening of the passageway, suitable for connection of an enteral feeder such as an enteral-only syringe or an enteral feeding extension set. The body also has an outlet segment including a stem projecting from the inlet segment and a frustoconical tip defining an outlet opening of the passageway, for connecting to an enteral feeding tube or an enteral feeding extension set tube. The enteral connector also includes a cap having an external cover for covering the outside of the inlet segment and a plug integral with and extending from the external cover for inserting into the inlet opening. The cap is connected to and integral with the body by an intermediate flexible neck.

According to another aspect, there is provided an enteral feeding device that includes a connector and an enteral tube extending from the connector. The connector includes a body defining a passageway therethrough. The body has an inlet segment defining an inlet opening of the passageway, suitable for connection of an enteral feeder such as an enteral-only syringe or an enteral feeding extension set. The body also has an outlet segment including a stem projecting from the inlet segment and a frustoconical tip defining an outlet opening of the passageway and connected to the enteral feeding tube. The enteral connector also includes a cap having an external cover for covering the outside of the inlet segment and a plug integral with and extending from the external cover for inserting into the inlet opening. The cap is connected to and integral with the body by an intermediate flexible neck.

Advantageously, the enteral connector includes a cap that is connected to the body by an intermediate flexible neck that is integral with the body and the cap, thereby providing a unitary part. Thus, without breaking the connector, detachment or loss of the cap is avoided. The cap of the connector includes both an external cover for covering and protecting the outside of the inlet segment of the body of the connector, and a plug that is integral with and extends from the external cover, for inserting into the inlet opening of the body of the connector. The cap thereby provides internal and external protection for the inlet segment of the body. Further, the external cover and the plug are sized and shaped to maintain the shape of the inlet segment of the body, thereby inhibiting deformation of the inlet segment.

In a particular aspect, the outlet segment of the connector includes the stem and the frustoconical tip with a lip at the stem side of the tip. The frustoconical tip and lip aid in connecting and maintaining connection to, for example a silicone feeding tube. In another particular aspect, the connector is a phthalate free polyvinyl chloride that is injection molded to provide a connector with suitable physical properties including flexibility and rigidity and while permitting connection to a variety of materials such as silicone or polyurethane tubes. A flange located intermediate the inlet segment and the outlet segment provides an abutment surface against which the enteral feeding tube abuts when inserted into the outlet opening defined by the outlet segment of the connector.

### BRIEF DESCRIPTION OF THE DRAWINGS

The present invention will be better understood with reference to the drawings and to the following description in which:

FIG. 1 is a perspective view of an enteral connector according to one embodiment of the present invention;

FIG. 2 is a sectional side view of the enteral connector of FIG. 1;

FIG. 3 is an alternative perspective view of the enteral connector of FIG. 1, with portions of the connector shown in section for the purpose of illustration;

FIG. 4 is sectional side view of a portion of the enteral connector of FIG. 1, the enteral connector shown connected to tube;

FIG. 5 is a sectional side view of a portion of the enteral connector of FIG. 1, the enteral connector shown connected to another tube;

FIG. 6 is a sectional side view of a portion of the enteral connector of FIG. 1, showing a cap covering an inlet segment of the enteral connector;

FIG. 7 is a perspective view of the enteral connector of FIG. 1 in use;

FIG. 8 is a perspective view of enteral connectors used in an enteral feeding extension set;

FIG. 9 is a side view of the enteral feeding extension set of FIG. 8, in use.

### DETAILED DESCRIPTION OF THE PREFERRED EMBODIMENTS

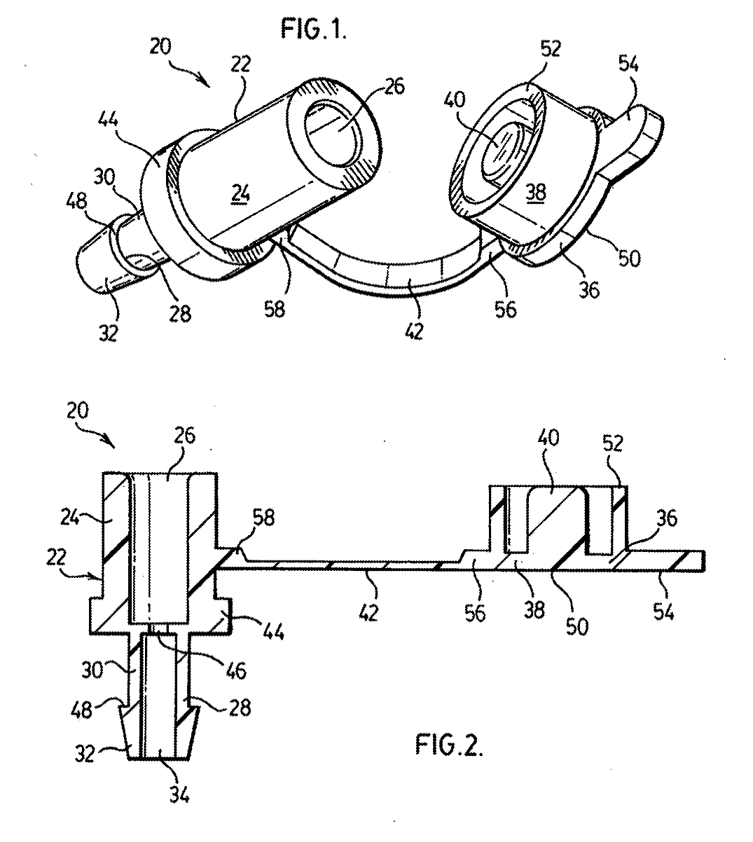

Reference is first made to FIGS. 1 and 2 to describe an enteral connector according to one aspect, the enteral connector being indicated generally by the numeral 20. The enteral connector 20 includes a body 22 defining a passageway therethrough. The body 22 has an inlet segment 24 defining an inlet opening 26 of the passageway, suitable for connection of an enteral feeder such as an enteral-only syringe or an enteral feeding extension set. The body 22 also has an outlet segment 28 including a stem 30 projecting from the inlet segment 26 and a frustoconical tip 32 defining an outlet opening 34 of the passageway, for connecting to an enteral feeding tube or to an enteral feeding extension set tube. The enteral connector 20 also includes a cap 36 having an external cover 38 for covering the outside of the inlet segment 24 and a plug 40 integral with and extending from the external cover 38 for inserting into the inlet opening 26. The cap 36 is connected to and integral with the body 22 by an intermediate flexible neck 42.

Continued reference is made to FIGS. 1 and 2 to describe the enteral connector 20 according to one embodiment, in detail. As will be appreciated, the enteral connector 20 is used for connecting at one end to an enteral feeding tube or to an enteral feeding extension set tube and at the other end for connecting an enteral feeder such as an enteral-only syringe or an enteral feeding extension set for delivering liquid feed through the enteral connector 20 and the enteral feeding tube. In the present embodiment, the enteral connector 20 is a single, unitary piece, injection molded from a phthalate-free polyvinyl chloride (PVC). The phthalate-free PVC is suitable for connecting to both silicone and polyurethane tubes. Further, phthalate-free PVC provides suitable rigidity and flexibility for use.

As indicated above, the enteral connector 20 includes a body 22 and a cap 36 that are joined together by an intermediate flexible neck 42. The body 22 includes an inlet segment 24 and an outlet segment 28 that are separated by a flange portion 44. The inlet segment 24 is cylindrically shaped and is hollow with an inlet opening 26 of the passageway at one end thereof. The opposite end of the inlet segment abuts the flange portion 44. The internal diameter of both the inlet segment 24 and the inlet opening 26 is sized for enteral-only connection of the enteral feeder such as an enteral feeding set or enteral-only syringe. Thus, the internal diameter of the inlet segment 24 and the inlet opening 26 is 4.70 mm (0.185 inches). The inlet opening 26 is sized to inhibit connection of a male luer slip to the inlet segment 24. Thus, the inlet opening 26 is larger than a standard male luer slip so that firm connection is not maintained. The external diameter of the inlet segment 24 is 8.89 mm (0.350 inches). The external diameter is sized to inhibit connection by a sleeve of a male luer lock. The external diameter is larger than the sleeve of a male luer lock connector.

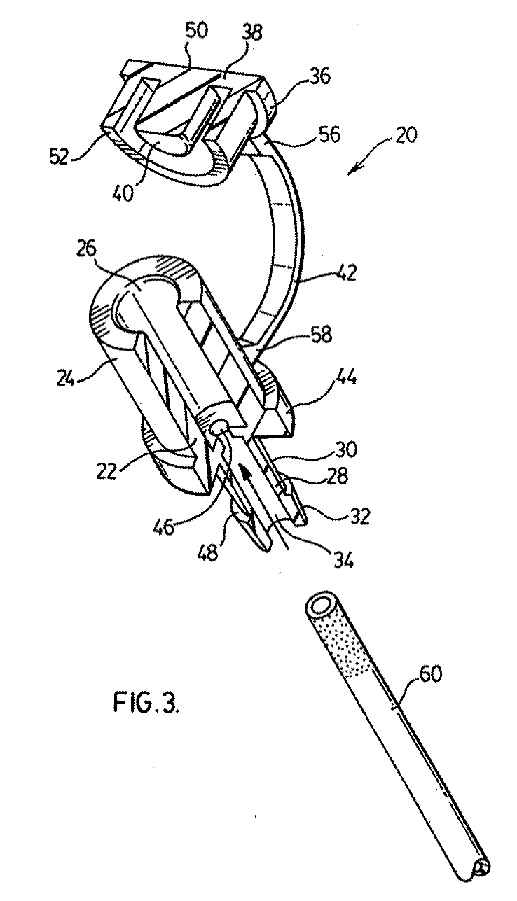

As described, the inlet segment 24 abuts the flange portion 44. The flange portion 44 is disc-shaped and includes a hole 46 through the middle thereof to form a part of the passageway that extends through the flange portion 44. As best shown in FIG. 3, the hole 46 is smaller in diameter than the internal diameter of the inlet segment 24.

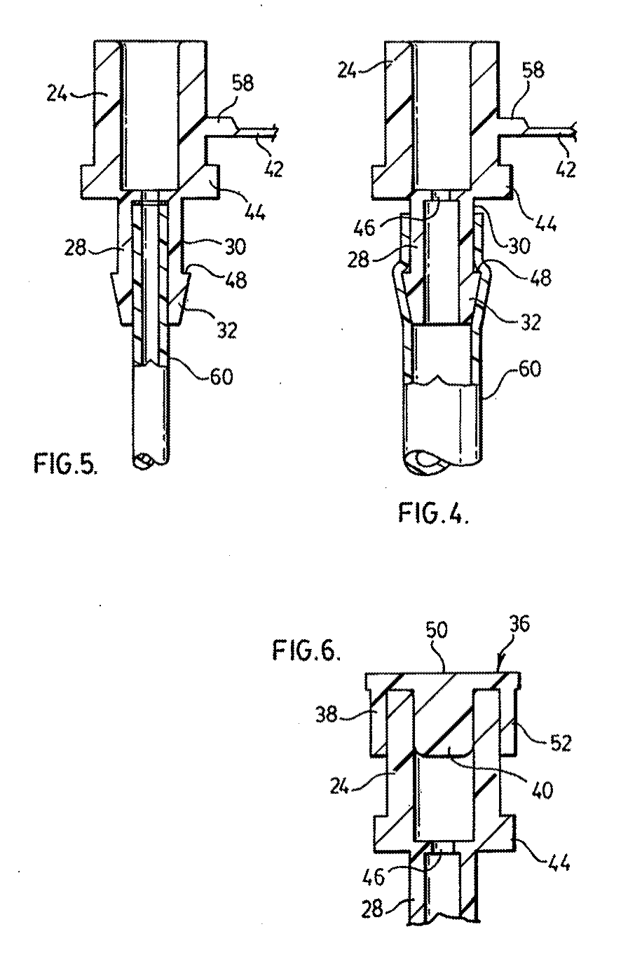

Referring now to FIG. 4, the outlet segment 28 is also hollow and is integral with and extends from the flange portion 44. As referred to above, the outlet segment 28 includes a stem 30 that joins to a tip 32. The stem 30 is frustoconically shaped with an outer diameter that decreases slightly with distance from the flange portion 44. Thus, the outer diameter of the stem 30 is largest immediately adjacent the flange portion 44. The tip 32 is also frustoconically shaped with an outer diameter that decreases with distance from the stem 30. Thus, the outer diameter of the tip 32 is larger immediately adjacent the stem 30. As shown, the outer diameter at the portion of the tip 32 that is immediately adjacent the stem 30, is larger than the outer diameter at the portion of the stem 30 that is immediately adjacent the tip 32. A lip 48 is thereby formed at the end of the frustoconical tip 32 that is adjacent the stem 30. The frustoconical tip 32 and lip 48 aid in connecting and maintaining connection to an enteral tube. For example, when used with a silicone enteral feeding tube, the end of the enteral feeding tube slips over the frustoconical tip 32 and lip 48 which serve to maintain the tube in place without the use of an adhesive.

The internal diameter of both the frustoconical tip 32 and the stem 30 is consistent and is larger than the diameter of the hole 46 through the flange portion 44. The internal diameter of the tip 32 and stem 30 is sized for connection to, for example, a polyurethane enteral feeding tube or enteral feeding extension set tube by insertion of the tube inside the tip 32 and stem 30 until the end of the tube abuts the flange portion 44, as shown in FIG. 5. The inlet segment 24, the flange portion 44 and the outlet segment 28 are all co-axial and together form the passageway through the body 22.

The cap 36 includes an external cover 38 that has an end wall 50 for abutting the end of the inlet segment 24 when the cap 36 covers the portion of the inlet segment 24 that is closest to the inlet opening 26, and a cylindrical extension 52 that is sized to snugly fit over the outside of the inlet segment 24, as shown in FIG. 6. Thus, the internal diameter of the cylindrical extension is 9.40 mm (0.370 inches) which is slightly larger than the external diameter of the inlet segment 24 to provide a snug fit. The cap 36 also includes a solid cylindrical plug 40 that is integral with and extends from the end wall 50, in the same direction and to about the same distance from the end wall 50 as the cylindrical extension 52. The plug 40 is sized to snugly fit into the inlet opening 26. Thus, the diameter of the plug 40 is approximately 4.70 mm (0.185 inches), which is approximately equal to the internal diameter of the inlet segment 24 to provide a snug fit. A tab 54 (best shown in FIGS. 1 and 2) extends outwardly from the end wall 50 of the cap 36, in the direction that is generally perpendicular to the axis of the cylindrical extension 52, for grasping by the user when removing the cap 36 from the body 22.

As indicated above, the cap 36 is joined to an integral with the body 22 by the intermediate flexible neck 42. The neck 42 is a thinned portion of suitable length to allow the cap 36 to be placed on and cover the portion of the inlet segment 24 at the inlet opening 26 and to be removed from the inlet segment 24. The neck 42 is connected at one end 56 to the end wall 50, on an opposite side as the tab 54. At the other end 58, the neck 42 is connected to the outside of the inlet segment 28, at a distance from the inlet opening 26 so as not to block the placement of the cap 36. A portion at each of the ends 56, 58 of the neck 42 is slightly larger in thickness to provide reinforcement where the neck joins to the cap 36 and to the body 22.

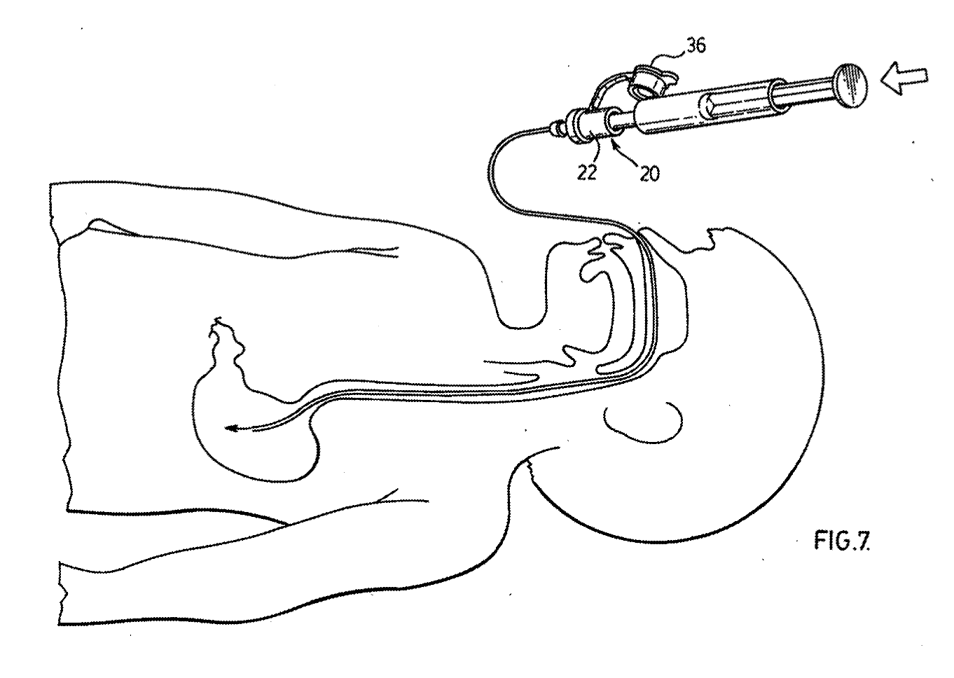

In one particular use, an enteral feeding tube is inserted into the outlet opening 34 of the enteral connector 20 and is fixed in place using a suitable solvent for adhering. In the present example, cyclohexanone is used. With the enteral tube in place in a patient as shown in FIG. 7, the cap 36 is removed from the body 22 and an enteral-only syringe, for example, is pushed into the inlet opening and depressed to deliver liquid feed from the syringe, through the connector 20 and through the enteral feeding tube, into the patient.

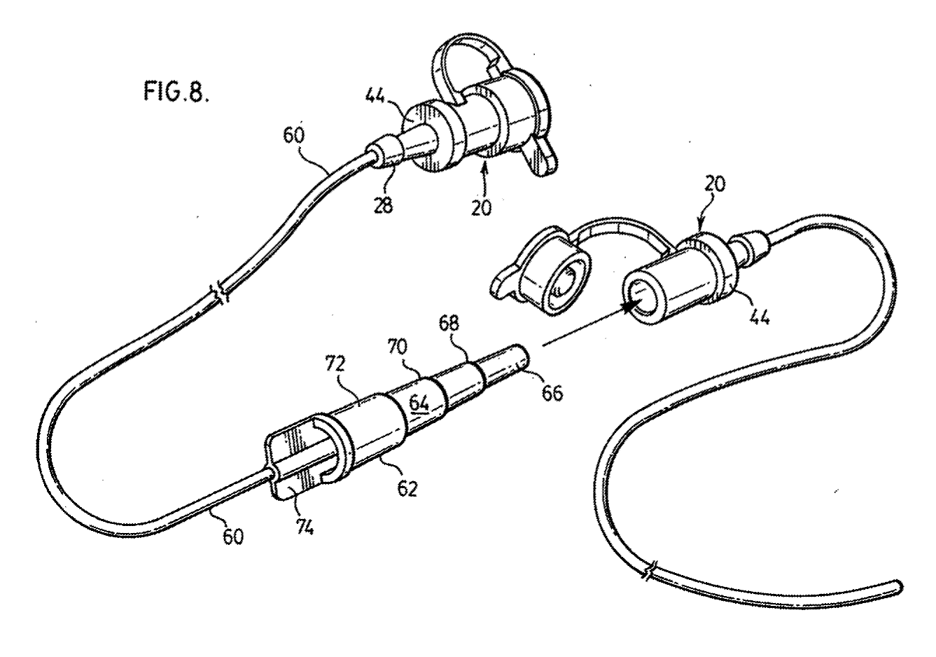

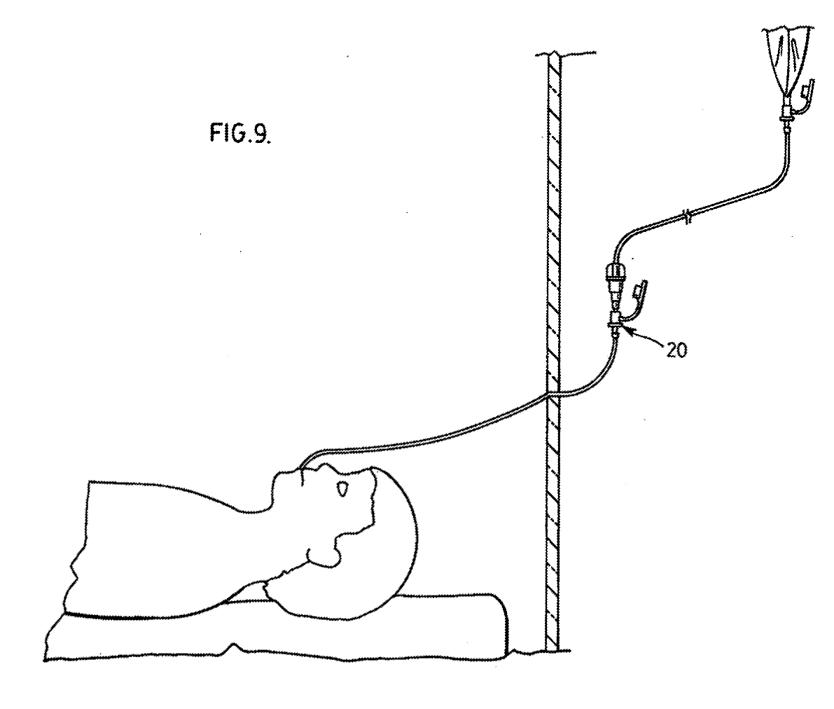

Reference is now made to FIGS. 8 and 9 to describe an enteral feeding extension set including a connector according to a second embodiment. According to the present embodiment, the enteral feeding extension set includes the enteral connector 20 which is fixed to an enteral feeding extension set tube 60. The enteral connector 20 is similar to that of the first-described embodiment and thus the details of the enteral connector 20 are described above. The tube 60 is fixed in the outlet segment 28 of the enteral connector 20 by, for example, cyclohexanone. The end of the tube 60 abuts the flange portion 44.

At the distal end of the enteral feeding extension set tube 60, an enteral only connector nozzle 62 is employed for fitting into an enteral-only connector, similar to the enteral connector 20, on an enteral feeding tube. The connector nozzle 62 includes a body 64 hollow cylindrical delivery end 66 with an opening that is in fluid connection with the tube 60 for delivery of the feed liquid. The body 66 also includes three cylindrical segments 68, 70, 72, each segment 68, 70, 72 increasing in diameter in stepwise fashion with distance from the delivery end 66. Thus, the first segment 68 is larger in diameter than the delivery end 66, the second segment 70 is larger in diameter than the first segment 68, and the third segment 72 is larger in diameter than the second segment 70. The stepwise increasing diameter of the body 64 allows for connection of the connector nozzle 62 to different sized connectors. A web 74 extends across the internal diameter of the body 64 and includes a conduit through which the tube 60 extends and to which the tube 60 is fixed using a suitable adhesive. Clearly the web 74 centers the tube 60 in the enteral-only connector nozzle 62 and provides a surface to which the tube 60 is secured.

While the embodiments described herein are directed to particular implementations of the present invention, it will be understood that modifications and variations to these embodiments are within the scope and sphere of the present application. For example, the size and shape of many of the features of the above-described embodiments can vary while still performing the same function. Although the embodiments above have been described with reference to Figures that show a polyurethane feeding tube, it will be appreciate that a silicone feeding tube can also be used as indicated above. The silicone feeding tube fits over the outlet segment 28 and is retained by the frustoconical tip 32. Many other modifications and variations may occur to those skilled in the art. All such modifications and variations are believed to be within the sphere and scope of the present application.

1. An enteral connector comprising:
a body having only a single passageway therethrough, the body comprising:
a single cylindrical inlet segment defining one inlet opening of the passageway, suitable for connection of an enteral feeder, an outer diameter of the inlet segment sized larger than a sleeve of a male luer lock connector to inhibit connection of a male luer lock thereto and a diameter of the inlet opening sized to inhibit connection of a male luer slip; and
an outlet segment comprising a stem projecting from the inlet segment and a frustoconical tip defining an outlet opening of the passageway, for connecting to an enteral feeding tube or to a tube of an enteral feeding extension, a diameter at a stem side of the frustoconical tip being larger than a diameter of the stem, to form a lip on the stem side of the frustoconical tip; and
a cap comprising an external cover for covering the outside of the inlet segment and a plug integral with and extending from the external cover for inserting into the inlet opening, the cap being connected to and integral with the body by an intermediate flexible neck.

2. (canceled)

3. The enteral connector according to claim 1, comprising a flange intermediate the inlet segment and the outlet segment.

4. The enteral connector according to claim 1, wherein the inlet segment is cylindrical.

5. (canceled)

6. The enteral connector according to claim 4, wherein a diameter of the inlet opening is about 4.70 mm.

7. The enteral connector according to claim 1, wherein a diameter of the outlet opening is about 4.445 mm.

8. The enteral connector according to claim 1, wherein the body and the cap are phthalate-free polyvinyl chloride.

9. (canceled)

10. The enteral connector according to claim 4, wherein the inlet segment is sized for connection of an enteral-only feeding set.

11. An enteral feeding device comprising:
a connector and an enteral feeding extension set tube extending from the connector, the connector comprising:
a body having only a single passageway therethrough, the body having a single cylindrical inlet segment defining one inlet opening of the passageway, suitable for connection of an enteral feeder, an outer diameter of the inlet segment sized larger than a sleeve of a male luer lock connector to inhibit connection of a male luer lock thereto, a diameter of the inlet opening sized to inhibit connection of a male luer slip, and an outlet segment comprising a stem projecting from the inlet segment and a frustoconical tip defining an outlet opening of the passageway and connected to the enteral feeding extension set tube, a diameter at a stem side of the frustoconical tip being lamer than a diameter of the stem, to form a lip on the stem side of the frustoconical tip; and a cap having an external cover for covering the outside of the inlet segment and a plug integral with and extending from the external cover for inserting into the inlet opening, the cap being connected to and integral with the body by an intermediate flexible neck.

12. (canceled)

13. The enteral feeding device according to claim 11, comprising a flange intermediate the inlet segment and the outlet segment.

14. The enteral feeding device according to claim 11, wherein the inlet segment is cylindrical.

15. (canceled)

16. The enteral feeding device according to claim 14, wherein a diameter of the inlet opening is about 4.70 mm.

17. The enteral feeding device according to claim 11, wherein a diameter of the outlet opening is about 4.445 mm.

18. (canceled)

19. The enteral feeding device according to claim 14, wherein the inlet segment is sized for connection of an enteral-only feeding set.

20. The enteral feeding device according to claim 11, comprising an enteral-only connector nozzle fixed at a distal end of said enteral feeding extension set tube.

21. The enteral feeding device according to claim 11, wherein the cap comprises a cylindrical extension sized to fit over an outside of the inlet segment.

22. The enteral feeding device according to claim 1, wherein the cap comprises a cylindrical extension sized to fit over an outside of the inlet segment.
